(19) United States
(12) Patent Application Publication
(10) Pub. No.: US 2026/0144878 A1
(43) Pub. Date: May 28, 2026

(54) STABLE OPHTHALMIC EMULSION

(71) Applicant: Alcon Inc., Fribourg (CH)

(72) Inventors: Alexei Grigoryevich Merzlikine, Benbrook, TX (US); Jonathan Matthew Li, Irving, TX (US); Mohammad Mostafa Kamal, Irving, TX (US); Ye Hong, Flower Mound, TX (US)

(21) Appl. No.: 19/400,407

(22) Filed: Nov. 25, 2025

Related U.S. Application Data
(60) Provisional application No. 63/726,075, filed on Nov. 27, 2024.

(51) Int. Cl. A61K 47/36; A61K 9/00; A61K 9/107; A61K 47/02; A61K 47/14; A61K 47/18; A61K 47/24; A61K 47/26; A61K 47/34

(52) U.S. Cl. CPC A61K 47/36; A61K 9/0048; A61K 9/107; A61K 47/02; A61K 47/14; A61K 47/186; A61K 47/24; A61K 47/26; A61K 47/34

(57) ABSTRACT

The present invention is directed to an ophthalmic emulsion. The emulsion has a unique combination of ingredients that promotes the thermal stability of small oil droplets within the emulsion. The emulsion also includes a mucoadhesive polymer that aid in delivering a lipid to the ocular surface.

### TECHNICAL FIELD OF THE INVENTION

The present invention is directed to an ophthalmic emulsion. More particularly, the present invention is directed to an ophthalmic emulsion having a unique combination that promotes the stability of the emulsion and promotes the therapeutic delivery capability of the emulsion.

### BACKGROUND OF THE INVENTION

There are a variety of types of ophthalmic compositions such as aqueous solutions, aqueous suspensions and emulsions.

Ophthalmic aqueous solution compositions for topical application, and in particular artificial tear compositions, comprise compounds that lubricate and protect the ocular surface. In the context of dry eye disorders, artificial tear compositions can prevent symptoms such as pain and discomfort and can prevent bioadhesion and tissue damage induced by friction. A large number of potential compounds are available that are useful as lubricants and ocular surface protectants. For example, certain marketed artificial tear products contain natural polymers such as galactomannans. Other lubricants and ocular surface protectants include, for example, carboxymethylcellulose, glucomannan, glycerol, and hydroxypropylmethylcellulose. As noted above, ophthalmic compositions have been previously described that utilize galactomannan compounds such as guar. U.S. Pat. No. 6,403,609 to Asgharian, entitled “Ophthalmic compositions containing galactomannan polymers and borate,” describes such systems and is herein incorporated by reference in its entirety.

Hyaluronic acid (HA) occurs naturally in the human body and Sodium hyaluronate is the water-soluble salt form of hyaluronic acid, a biocompatible polysaccharide with unique hygroscopic and viscoelastic properties. as a natural lubricant and its water-retaining properties make it well-suited for the ocular surface to help provide enhanced hydration and comfort to dry eye patients. Sodium hyaluronate has been shown to effectively treat symptoms of dry eye. Sodium hyaluronate eye drops increase precorneal tear film stability and corneal wettability, reduce the tear evaporation rate, and the healing time of corneal epithelium (Aragona P, Di Stefano G, Ferreri F, et al. Sodium hyaluronate eye drops of different osmolarity for the treatment of dry eye in Sjogren's syndrome patients. Br J Ophthalmol 2002 August; 86 (8): 879-84).

The combination of sodium hyaluronate and hydroxypropyl guar in a single aqueous solution formulation provided an effective moisture layer that resulted in significantly greater cell viability after desiccation than either polymer alone, and protection from desiccation was evident even after test solutions were rinsed away. U.S. patent Ser. No. 10/828,320 to Davis et. Al. entitled “Ophthalmic Compositions With Improved Dessication Protection And Retention” describes such systems and is herein incorporated by reference in its entirety.

Contrast to above ophthalmic aqueous solution compositions, ophthalmic compositions are also formulated as emulsions. Ophthalmic emulsions are typically employed in circumstances where it is desirable to include two or more ingredients that are immiscible relative to each other in a single composition and therefore form two separate phases within the composition. Such emulsions can allow a single composition to provide the advantages attributable to both phases (e.g., advantageous delivery characteristics). For example, an emulsion can be formed of oil droplets in an aqueous phase where the oil droplets can be used as carriers for actives such as therapeutic agents (e.g., drugs) or excipients which have poor solubility and/or stability in water. Examples of emulsions are included in U.S. Pat. Nos. 4,914,088; 5,278,151; 5,294,607; 5,371,108; and 5,578,586. Each of these patents is incorporated herein by reference for all purposes.

It is typically quite desirable for one phase of an emulsion to be substantially uniformly dispersed within the other phase. Such dispersion can significantly effect the capabilities of emulsion to deliver therapeutic ingredients. Moreover, such dispersion is often an indication of the stability of the emulsion itself.

The separate phases of an emulsion can be extremely difficult to evenly disperse throughout a composition since each phase tends to associate with itself rather than the other phase. Thus, the maintenance of the distribution of one phase (i.e., the dispersed phase) within the other phase (i.e., the continuous phase) can be very delicate. Moreover, it is also often difficult to include additional ingredients within an emulsion since many ingredients can act to inhibit the dispersion and/or even distribution of the dispersed phase throughout the continuous phase.

The ophthalmic emulsion compositions have been previously described that utilize galactomannan compounds such as guar. U.S. patent Ser. No. 10/004,685 to Ketelson entitled “Ophthalmic Emulsion,” describes such emulsion systems and is herein incorporated by reference in its entirety. However, it is difficult to incorporate water soluble polymers such as HA into ophthalmic oil-in-water emulsions.

Accordingly, there is a need for an ophthalmic emulsion containing both galactomannan compounds and HA polymer and ophthalmic emulsion is a stable emulsion offering resistance to cream forming and phase separation during storage. There is also a need to provide a process that is capable producing a stable emulsion containing both galactomannan compounds and HA polymer. The ophthalmic emulsion eye drops coat and protect the surface of the eye, while providing hydration and lubrication

### SUMMARY OF THE INVENTION

1. The present invention is directed to A thermal stable ophthalmic emulsion, the emulsion comprising:
   water forming an aqueous phase;
   oil forming an oil phase, wherein oil in amount from 0.8 to 1.2 w/v %; a charged phospholipid;
   two surfactants, a first surfactant is a hydrophilic surfactant having an HLB value of from 10 to 18, a second surfactant is a hydrophobic surfactant having an HLB value of from 1 to 6, and a combination thereof, wherein the first surfactant is not the charged phospholipid;
   borate;
   two mucoadhesive polymers, the first mucoadhesive polymer is sodium hyaluronate, the second mucoadhesive polymer is galactomannan polymer; wherein a ratio of the total concentration of the two mucoadhesive polymers to the total concentration of two surfactants is between 0.20 and 0.40;
   Wherein a ratio oil to the total concentration of two surfactants is between 0.6 and 1.1;
   wherein the thermally stable ophthalmic emulsion increase of D90 less than 40% after 6 freeze-thaw cycles
   The borate and galactomannan polymer cooperatively act to form a gel upon instillation of the emulsion in an eye of an individual.

The present invention is also directed to a thermal stable ophthalmic emulsion, the emulsion consisting essentially of or consisting of:
water forming an aqueous phase;
oil forming an oil phase, wherein the oil in amount from 0.8 to 1.2 w/v %;
an ionic charged phospholipid, wherein the ionic charged phospholipid in amount of 0.005 w/v %;
a hydrophilic surfactant, wherein the hydrophilic surfactant is polyoxyethylene-40-stearate and in amount of 0.5 w/v % to 0.8 w/v %.
a hydrophobic surfactant, wherein the hydrophobic surfactant is sorbitan tristearate and in amount of 0.4 w/v % to 0.6 w/v %.
borate, wherein the borate is a boric acid in amount of 1.0 w/v %;
sodium hyaluronate, wherein the sodium hyaluronate is in amount of 0.15 w/v %;
galactomannan polymer, wherein the galactomannan polymer is in amount of 0.15 w/v
wherein the thermally stable ophthalmic emulsion increase of D90 less than 40% after 6 freeze-thaw cycles.

### DETAILED DESCRIPTION OF THE INVENTION

The present invention is predicated upon the provision of an ophthalmic oil in water emulsion. The emulsion will typically be aqueous and include a substantial amount of water. The emulsion will also typically include an anionic phospholipid, a hydrophilic surfactant (high HLB) and a hydrophobic (low HLB) surfactant. Further, the emulsion will typically include two high molecular weight mucoadhesive ingredients (e.g., galactomannan polymers and Hyaluronic acid polymer) to aid in maintaining the emulsion on the corneal surface of the eye and/or aid in delivering one or more lipophilic compounds to the corneal surface. The emulsions of the present invention are most desirably used for dry eye therapeutics. However, without limitation, it is also contemplated that the emulsions may be used for drug delivery, vitamin delivery, botanical delivery, contact lens wetting and contact lens lubrication.

The present invention is partly based on the finding that the increased amount of water soluble high molecular weight polymers (such as hydroxypropyl guar and HA) affects the stability of the emulsion, which is observed as creaming during storage and as increased creaming rate. An oil-in-water emulsion is usually generated and stabilized by a surfactant emulsifier. High molecular weight polymers (such as hydroxypropyl guar and HA) destabilize oil-in-water emulsions via the Bridging Flocculation or Depletion Flocculation effects. The water soluble high molecular weight polymer interacts with the emulsion droplets forming a cluster of emulsion-polymer complexes. When too many emulsion droplets are picked up by the polymer, the overall mass of the cluster becomes so large that the buoyancy overcomes the Brownian motion and creaming occurs.

The present invention is also partly based on the finding that the increased amount of surfactants affects the stability of the emulsion, which is observed as creaming during storage and as decreases creaming rate. However, it should be understood that with many Surfactants, as concentration increases, the likelihood of physical discomfort—i.e., stinging, of the emulsion on the eye increases. Surfactants can irritate the eyes because of their surface active properties and ability to disrupt lipid membranes that protect cells. Thus, if significant stinging occurs when the emulsion is applied to the ocular surface, it is likely that the concentration of Surfactant is too high. The present invention is partly based on the finding that if the emulsion of the present invention has an emulsion having a ratio of the total concentration of the oil and two surfactants to the total concentration of the two high molecular weight mucoadhesive polymers is a ratio oil to the total concentration of two surfactants is between 0.6 and 1.1 and a ratio of the total concentration of the two mucoadhesive polymers to the total concentration of two surfactants is between 0.20 and 0.40, the emulsion is stable as observed as creaming during storage and as decreases creaming rate is lower than 0.5 mm/day. if the emulsion of the present invention having a ratio of the total concentration of the two mucoadhesive polymers to the total concentration of two surfactants is less than 0.20 such as 0.15, the emulsion is stable but the emulsion may cause the likelihood of physical discomfort-such as stinging, on the eye increases.

The present invention is further partly based on the finding that if the emulsion of the present invention has an emulsion having a ratio of the total concentration of the oil and two surfactants to the total concentration of the two high molecular weight mucoadhesive polymers is a ratio oil to the total concentration of two surfactants is between 0.6 and 1.1 and a ratio of the total concentration of the two mucoadhesive polymers to the total concentration of two surfactants is between 0.20 and 0.40, the emulsion is the thermally stable as observed as the ophthalmic emulsion increase of D90 less than 40% after 6 freeze-thaw cycles.

Freeze-thaw cycling is a stress test method for emulsions. As the sample freezes, water soluble components are concentrated in the liquid phase, and may increase flocculation of the emulsion or destabilize it. The ice crystals push the oil droplets together and/or mechanically disrupt the surface layer of the oil droplets, causing irreversible droplet coalescence and eventually oil/water phase separation and results in increasing of D90.

Lumisizer instrument is a dispersion analyzer that predicts the emulsion stability by analyzing light transmission creaming rate across the sample in a cell subjected to high centrifugal forces under various temperatures. It measures the changes in light transmission caused by centrifugation versus time and the position in the vial and translates them into and other parameters. To measure the creaming rate of an emulsion using a Lumisizer instrument, you need to fill a sample cell with your emulsion, place it in the Lumisizer, and centrifuge it while monitoring the change in light transmission across the sample height over time; the software will analyze the transmission profile to calculate the creaming velocity, which is directly related to the creaming rate of the emulsion. The creaming rate we measure extrapolated to 1 G gravity is 0.07 mm/day for best case scenario and 0.2-0.5 mm/day for what is still good.

The present invention is still further partly based on the finding that addition sodium hyaluronate (HA) into a control oil-in-water emulsion results in a new (invention) oil-in-water emulsion, wherein the new oil-in-water emulsion has a reduced evaporative flux, wherein the control oil-in-water emulsion and the new oil-in-water emulsion have the exactly the same ingredients except of the latter containing sodium hyaluronate. This result suggests that sodium hyaluronate may may facilitate the formation of a more efficient barrier by oil in oil-in-water emulsion for evaporation from the aqueous interface. This reduced evaporative flux demonstrates the effectiveness of eye-drop formulations in reducing tear evaporation, which is important for managing dry eye.

A form a bridged matrix to synergistically relieve the symptoms of dry eye. HA is an effective ocular lubricant that helps support tear film stability.

Unless otherwise specifically stated all emulsion ingredient amounts or percentages are weight volume percentages (w/v %).

The oil of the emulsion is dispersed throughout the continuous water or aqueous phase as small droplets that are substantially distinct and separate. It should be understood that, as used herein, the phase distinct and separate means that, at any give point in time, the droplets are distinct and separate. However, the droplets of the emulsion can combine and separate over time to maintain an average droplet size or diameter. The droplets of the emulsion of the present invention typically have an average or mean diameter no greater than about 300 nanometers (nm), more typically no greater than about 200 nm and still more typically no greater than about 120 nm. These droplets also typically have an average or mean diameter that is typically at least 2 nm, more typically at least 10 nm and still more typically at least 50 nm.

According to the present application, the oil phase is in droplets within the aqueous phase and the droplets have a D90 diameter that is no greater than 120 nm but is at least 50 nm; determined using Anton Paar Litesizer 500 DLS as the droplet diameter corresponding to 90% of the cumulative undersize distribution by volume”

Particle size analyzers may be used to determine emulsion oil droplet size. For example, Anton Paar Litesizer 500 is a dynamic light scattering (DLS) device that can be used to measure the emulsion oil droplet size and droplet size distribution. DLS derives particle size information from the time dependence of the scattered laser radiation intensity that passes through the emulsion. DLS software optimizes the measurement conditions, such as the detection angle, and uses the time dependence of the scattered intensity (autocorrelation function) to calculate the average size of the particles (hydrodynamic radius) as well as the size distribution. The droplet diameter corresponding to 90% of the cumulative undersize distribution by intensity is used.

Assessing whether there has been a change in the particle size distribution of the emulsion over time is a good measure of emulsion stability. A lack of change, or a small change, in particle size distribution of the emulsion overtime, indicates the emulsion is stable. As used herein, a particle size “distribution” refers to the number or concentration (e.g., percentage) of particles having a certain size (i.e., diameter), or range of sizes, within a given emulsion, lot and/or batch of the resent invention. As used herein, a particle size “distribution” refers to the number or concentration (e.g., percentage) of particles having a certain size (i.e., diameter), or range of sizes, within a given emulsion, lot and/or batch of the present invention

As used herein, a “D50” or “d(0.5)” value refers to the particle size of an oil phase, and specifically the diameter at which 50% of the measurable particles of the oil phase particles have a larger equivalent diameter, and the other 50% of the particles have a smaller equivalent diameter. Thus, D50 generally refers to the median particle diameter.

As used herein, a “D90” or “d(0.9)” value refers to the particle size of an oil phase, and specifically the diameter at which 90% of all measurable particles of the oil phase have a diameter equal to or less than the D90 value, and 10% of the measurable particles have a diameter greater than the D90 value.

As used herein, “emulsion stability” refers to the ability of an emulsion to resist changes in the physical and chemical properties of the emulsion, including physical destabilization such as creaming, flocculation, coalescence, partial coalescence, phase inversion and Ostwald ripening over time.

The thermally stable oil-in-water emulsions of the present invention refers to the emulsions can be subjected to test freeze-thaw cycle without undergoing a substantial change in particle size. As used herein, a “freeze-thaw cycle” is conducted by exposing the oil-in-water emulsion are exposed to 3 cycles consisting of −20° C. for 28 hours, and then followed by 30° C. for 28 hours (1 week duration). In some embodiments, the thermally stable oil-in-water emulsions of the present invention are free from a variation in particle size for at least five or six freeze-thaw cycles, and preferably for at least 3 freeze-thaw cycles. In some embodiments, an oil-in-water emulsion of the present invention is free from a variation in particle size after 6 freeze-thaw cycles.

As used herein, a “variation in particle size” can refer to an increase in D90 of about 40% or more, about 30% or more, about 25% or more, about 20% or more, or about 10% or more.

Self emulsifying (or self microemulsifying) drug delivery systems (SEDDS or SMEDDS) are mixtures of oils, surfactants, solvents, and drug substance that rapidly and spontaneously form oil-in-water emulsions or microemulsions when introduced into aqueous phases under gentle agitation. However, the present invention is not formed by Self emulsifying or self microemulsifying method.

The emulsion of the present invention is an oil in water emulsion. The oil can be any of numerous mineral, vegetable, and synthetic substances and/or animal and vegetable fats or any combination of oils. The oil can be soluble in various organic solvents such as ether but not in water. The oil phase can comprise, if desired a liquid hydrocarbon, such as a mineral oil, paraffin oils, petrolatum or hydrocarbon oils. Mineral oil is particularly preferred. A silicone oil may also be used. The oil phase can additionally include a waxy hydrocarbon, such as paraffin waxes, hydrogenated castor oil, Synchrowax HRC, Carnabau, beeswax, modified beeswaxes, microcrystalline waxes, and polyethylene waxes.

According to the present invention, the amount of the oil is from about 0.01 to about 20 w/v %, about 0.1 to about 15 w/v %, about 0.1 to about 4 w/v %, about 0.1 to about 3 w/v %, about 0.1 to about 2 w/v %, about 0.5 to about 8 w/v %, about 0.5 to about 6 w/v %, about 0.5 to about 5 w/v %, about 0.5 to about 4 w/v %, about 0.5 to about 3 w/v %, about 0.5 to about 2 w/v %, about 0.8 to about 1.2 w/v %, about 0.8 to about 1.1 w/v %, about 0.9 to about 1.2 w/v %, about 0.9 to about 1.1 w/v %. In some embodiment, the amount of the oil is 1 w/v %.

The emulsion of the present invention also typically incorporates two surfactants, which act as emulsifiers aiding in the emulsification of the emulsion. Typically, these surfactants are non-ionic. The concentration of emulsifying surfactant in the emulsion is often selected in the range of from 0.1 to 10% w/v, and in many instances from 0.5 to 5% w/v. It is preferred to select a first emulsifier/surfactant which is hydrophilic and has an HLB value of at least 8 and often at least 10 (e.g., 10 to 18). It is further preferred to select a second emulsifier/surfactant which is hydrophobic and has an HLB value of below 8 and particularly from 1 to 6. By employing the two surfactants/emulsifiers together in appropriate ratios, it is readily feasible to attain a weighted average HLB value that promotes the formation of an emulsion. For most emulsions according to the present invention, the average HLB value is chosen in the range of about 6 to 12, and for many from 7 to 11.

For example:

For example, the HLB values for exemplary surfactants and mineral oil are as follows: hydrophobic surfactant (2.1), hydrophilic surfactant (16.9) and mineral oil (10.5). The concentrations of hydrophobic surfactant and hydrophilic surfactant used in exemplary emulsions were 0.38% and 0.29% based on these calculations.

| 58/1.34 = 0.43 and .76/1.34 = 0.57 | | |
|---|---|---|
| hydrophobic surfactant | 2.1 × 0.43= | 0.90 |
| hydrophilic surfactant | 16.9 × 0.57= | 9.63 |
| | | 10.53 |

The ratio between hydrophobic surfactant and hydrophilic surfactant is equal to 1.32 which can be used to select the proper ratio of concentrations to be used for the two surfactants. The concentrations of hydrophobic surfactant and hydrophilic surfactant used in exemplary emulsions were 0.38% and 0.29% based on these calculations.

Different oils have different HLB requirements. For example, vegetable oil emulsions need an emulsifier with an HLB of 7-8, whereas the required HLB value to form a stable mineral oil emulsion is 10. By matching the HLB value of the emulsifier with that of the oil, formulators can greatly increase their chances of producing a stable emulsion.

The hydrophilic surfactant is typically present in the emulsion in an amount between 0.15 w/v % to 2.0 w/v %, more typically between 0.35 w/v % to 1.5 w/v %, even more typically between 0.5 w/v % to 0.8 w/v %, still even typically 0.57 w/v %, still even typically 0.76 w/v %. According to the present invention, the hydrophilic surfactant does not include the charged phospholipid.

The hydrophilic surfactant can be a fatty acid, an ester, an ether, an acid or any combination thereof. The hydrophilic surfactant may be ionic or non-ionic, but is preferably non-ionic. Many suitable surfactants/emulsifiers are nonionic ester or ether emulsifiers comprising a polyoxyalkylene moiety, especially a polyoxyethylene moiety, often containing from about 2 to 80, and especially 5 to 60 oxyethylene units, and/or contain a polyhydroxy compound such as glycerol or sorbitol or other alditols as hydrophilic moiety. The hydrophilic moiety can contain polyoxypropylene. The emulsifiers additionally contain a hydrophobic alkyl, alkenyl or aralkyl moiety, normally containing from about 8 to 50 carbons and particularly from 10 to 30 carbons. Examples of hydrophilic surfactants/emulsifiers include ceteareth-10 to -25, ceteth-10-25, steareth-10-25, and PEG-15-25 stearate or distearate. Other suitable examples include C10-C20 fatty acid mono, di or tri-glycerides. Further examples include C18-C22 fatty alcohol ethers of polyethylene oxides (8 to 12 EO). One particularly preferred hydrophilic surfactant is polyoxyethylene-40-stearate, which is sold under the tradename MYRJ-52, which is commercially available from Nikko Chemicals.

The hydrophopbic surfactant is typically present in the emulsion in an amount between 0.15 w/v % to 2.0 w/v %, more typically between 0.35 w/v % to 1.5 w/v %, even more typically between 0.4 w/v % to 0.6 w/v %, still even typically 0.435 w/v %. still further even typically 0.58 w/v %. According to the present invention, the hydrophilic surfactant does not include the charged phospholipid.

The hydrophobic surfactant can be a fatty acid, an ester, an ether, an acid or any combination thereof. The hydrophobic surfactant may be ionic or non-ionic, but is preferably non-ionic. The hydrophobic surfactant will typically include a hydrophobic moiety. The hydrophobic moiety can be either linear or branched and is often saturated, though it can be unsaturated, and is optionally fluorinated. The hydrophobic moiety can comprise a mixture of chain lengths, for example those deriving from tallow, lard, palm oil sunflower seed oil or soya bean oil. Such non-ionic surfactants can also be derived from a polyhydroxy compound such as glycerol or sorbitol or other alditols. Examples of hydrophobic surfactants include, without limitation, sorbitan fatty acid esters such as sorbitan monoleate, sorbitan monostearate, sorbitan monolaurate, sorbitan monopalmitate, sorbitan monoisostearate, sorbitan trioleate, sorbitan tristearate, sorbitan sesquioleate, sorbitan sesquistearate, combinations thereof or the like. One particularly preferred hydrophobic surfactant is a sorbitan tristearate sold under the tradename SPAN-65, which is commercially available from Croda Worldwide.

The types of galactomannans that may be used in the present invention are typically derived from guar gum, locust bean gum and tara gum. As used herein, the term “galactomannan” refers to polysaccharides derived from the above natural gums or similar natural or synthetic gums containing mannose or galactose moieties, or both groups, as the main structural components. Preferred galactomannans of the present invention are made up of linear chains of (1-4)-.beta.-D-mannopyranosyl units with .alpha.-D-galactopyranosyl units attached by (1-6) linkages. With the preferred galactomannans, the ratio of D-galactose to D-mannose varies, but generally will be from about 1:2 to 1:4. Galactomannans having a D-galactose:D-mannose ratio of about 1:2 and the molecular weight ranges from 50,000 to 300,000 Da are most preferred.

Additionally, other chemically modified variations of the polysaccharides are also included in the “galactomannan” definition. For example, hydroxyethyl, hydroxypropyl and carboxymethylhydroxypropyl substitutions may be made to the galactomannans of the present invention. Non-ionic variations to the galactomannans, such as those containing alkoxy and alkyl (C1-C6) groups are particularly preferred when a soft gel is desired (e.g., hydroxylpropyl substitutions). Substitutions in the non-cis hydroxyl positions are most preferred. An example of non-ionic substitution of a galactomannan of the present invention is hydroxypropyl guar, with a molar substitution of about 0.4. Anionic substitutions may also be made to the galactomannans. Anionic substitution is particularly preferred when strongly responsive gels are desired.

A galactomannan is typically present in a formulation of the present invention at a concentration of at least about 0.005 w/v %, more typically at least about 0.01 w/v % and even more typically at least about 0.03 w/v %, but typically no greater than about 5 w/v %, more typically no greater than about 1.0 w/v %, still more typically no greater than about 0.3 w/v % and even still more typically no greater than about 0.08 w/v %. Compositions of the present invention comprise from about 0.05 to about 0.5 w/v % galactomannan. In a preferred embodiment, galactomannan is present at a concentration of about 0.1 to about 0.2 w/v %, and more preferably at a concentration of about 0.13 to 0.17 w/v %. In one embodiment, sodium hyaluronate is present at a concentration of about 0.15 w/v %.

Preferred galactomannans of the present invention are guar and hydroxypropyl guar.

Glycosaminoglycans such as hyaluronic acid are negatively charged molecules. Hyaluronic acid is an unsulphated glycosaminoglycan composed of repeating disaccharide units of N-acetylglucosamine (GlcNAc) and glucuronic acid (GlcUA) linked together by alternating beta-1,4 and beta-1,3 glycosidic bonds. Hyaluronic acid is also known as hyaluronan, hyaluronate, or HA. As used herein, the term hyaluronic acid also includes salt forms of hyaluronic acid such as sodium hyaluronate. Compositions of the present invention comprise from about 0.05 to about 0.5 w/v % hyaluronic acid. In a preferred embodiment, hyaluronic acid is present at a concentration of about 0.1 to about 0.2 w/v %, and more preferably at a concentration of about 0.13 to 0.17 w/v %. In one embodiment, sodium hyaluronate is present at a concentration of about 0.15 w/v %. A preferred hyaluronic acid is sodium hyaluronate. The molecular weight of the hyaluronic acid used in compositions of the present invention may vary, but is typically 0.5 to 2.0 M Daltons. In one embodiment, the hyaluronic acid has a molecular weight of 900,000 to 2.0 M Daltons. In another embodiment, the hyaluronic acid has a molecular weight of 0.8 to 2.0 M Daltons.

The emulsion may include additional or alternative polymeric ingredients and/or viscosity agents. Examples include, without limitation, carboxymethylcellulose, hydroxypropylmethyl cellulose, hydroxyethyl cellulose, carboxyvinyl polymer, xanthan gum, hyaluronic acid, any combinations thereof or the like.

The emulsion of the present invention includes at least one phospholipid for providing lipid over the ocular surface. The phospholipid composition in the form of an oil in water emulsion is topically applied to the eye where it is to disperse over the ocular surface and form a film that replicates a lipid layer that would be formed by the spreading of a naturally occurring lipid secreted principally from the Meibomian glands during blinking. The film formed from the charged phospholipid assists in the formation of a barrier film reducing evaporation of the aqueous layer, thereby preserving the tear film. In addition, phospholipid may aid in maintaining the stability of the emulsion and for reducing droplet size of the oil. It is known that complex phospholipids can contain a polar group at one end of their molecular structure and a non-polar group at the opposite end of their molecular structure. A discussion of phospholipids can be found in Lehninger, Biochemistry, 2 ed., Worth Publishers, New York, pp. 279-306, incorporated herein by reference for all purposes. According to the present application, phospholipid is defined as neither a surfactant nor an emulsifier.

Many complex phospholipids are known to the art. They differ in size, shape and the electric charge of their polar head groups. Phosphoglycerides are compounds where one primary hydroxyl group of glycerol is esterified to phosphoric acid, and the other two hydroxyl groups are esterified with fatty acids. The parent compound of the series is, therefore, the phosphoric acid ester of glycerol. This compound has an asymmetric carbon atom and, therefore, the term phosphoglycerides includes stereoisomers. All phosphoglycerides have a negative charge at the phosphate group at pH 7, and the pKa of this group is in the range of 1 to 2. The head groups of phosphatidylinositol, phosphatidylglycerol including diphosphatidylglycerols (having the common name cardiolipins) and the phosphatidylsugars have no electric charge, and all are polar because of their high hydroxyl group content. Because of the negative charge of the phosphate group and the absence of a charge in the head group, the net charge of each of these materials is negative, and these materials are within the scope of the invention. Suitable phospholipids are those carrying a net positive or negative charge under conditions of use. The preferred materials are those carrying a net negative charge because the negatively charged material will be repelled by the negatively charged ocular surface thereby permitting the maintenance of a relatively thick aqueous layer upon application to the eye. The most preferred phospholipid is an anionic phospholipid named dimyristoyl phosphatidylglycerol (DMPG), which is a polyol with a net negative charge. Phosphatidylglycerol or a phosphatidylinositol are other examples. Suitable phospholipid additives are disclosed in the above cited U.S. Pat. No. 4,914,088, which is fully incorporated herein by reference for all purposes.

Most phospholipids are limited water solubility. However, for application to the eye, it is desirable that the phospholipid be homogeneously distributed throughout an aqueous medium. For those few phospholipids having a solubility within a useful concentration range for use as a treatment composition, a simple aqueous solution of the phospholipid in saline is satisfactory. For those phospholipids that are essentially water insoluble, an aqueous composition in the form of an emulsion may be used. An emulsion provides a treatment composition where the phase containing the phospholipid component is homogeneously distributed throughout the aqueous vehicle.

The concentration of the phospholipid in the treatment composition may vary within wide limits. A treatment composition containing the complex phospholipid in an amount as low as 0.005 w/v % provides some benefit. When the treatment composition is in the form of an emulsion, compositions containing the phospholipid in elevated concentrations approaching collapse of the emulsion into separate aqueous and phospholipid phases is possible. A clinically practical concentration range for the phospholipid in its vehicle varies from about 0.005 to 7.0 w/v % phospholipid by weight, and more preferably varies from about 0.005 and 5.0 w/v %. In some embodiment of the present application, emulsion contains 0.005 w/v %. It should be noted that the most desired concentration for the phospholipid in the aqueous composition will vary from subject to subject.

Other additives may be present in the phospholipid treatment composition including neutral lipids such as one or more triglycerides, cholesterol esters, the natural waxes and cholesterol; higher molecular weight isoprenoids; stabilizers; preservatives; pH adjustors to provide a composition preferably having a pH between about 6 and 8 and more preferably between about 7.0 and 7.4; salt in sufficient concentration to form an isotonic composition; medicants; etc.

As indicated above, the emulsions of the present invention can include borate or borate/polyol buffer systems. As used herein, the term “borate” includes boric acid, salts of boric acid, other pharmaceutically acceptable borates, and combinations thereof. The following borates are particularly preferred: boric acid, sodium borate, potassium borate, calcium borate, magnesium borate, manganese borate, and other such borate salts.

As used herein, the term “polyol” includes any compound having at least one hydroxyl group on each of two adjacent carbon atoms that are not in trans configuration relative to each other. The polyols can be linear or cyclic, substituted or unsubstituted, or mixtures thereof, so long as the resultant complex is water soluble and pharmaceutically acceptable. Examples of such compounds include: sugars, sugar alcohols, sugar acids and uronic acids. Preferred polyols are sugars, sugar alcohols and sugar acids, including, but not limited to: mannitol, glycerin, xylitol and sorbitol. Especially preferred polyols are mannitol and sorbitol; most preferred is sorbitol.

The use of borate-polyol complexes in ophthalmic compositions is described in U.S. Pat. No. 6,503,497 (Chowhan); the entire contents of which are hereby incorporated in the present specification by reference. The emulsions of the present invention preferably contain one or more borates in a concentration that is at least about 0.01% w/v, more typically at least about 0.3% w/v and even more typically at least about 0.8% w/v, but typically no greater than about 5.0% w/v, more typically no greater than about 2.0% w/v and even more typically no greater than about 1.2% w/v. It is generally desirable for the amount of the one or more borates to be sufficient to allow the formation of borate/polyol complexes and, when desired, to aid in gelling the galactomannan polymer upon application of the emulsion to the eye.

The compositions of the present invention may not include a preservative. However, the compositions of the present invention may include a preservative. Potential preservatives include, without limitation, hydrogen peroxide, chlorine containing preservatives such as benzalkonium chloride or others. According to a preferred aspect, however, the ophthalmic composition of the present invention is substantially free of any chloride containing preservatives and, particularly, is substantially free of benzalkonium chloride. Benzalkonium chloride, as a mixture of alkyl dimethyl benzyl ammonium having various alkyl chain lengths is used as preservative agent in topical ophthalmic products. Benzalkonium chloride also has cationic agent properties, and was used as cationic agents for emulsions, especially ophthalmic emulsions. According to the present, the ophthalmic emulsion composition do not contain benzalkonium chloride preservatives and benzalkonium chloride emulsion agent (surfactant).

Most preferred preservatives included in the ophthalmic composition are polymeric quaternary ammonium compounds. Still another most preferred option, no preservatives are included in the ophthalmic composition.

As used herein, the phrase “substantially free of” as it refers to an ingredient of the ophthalmic composition means that it is contemplated that the ophthalmic solution can be either entirely devoid of that particular ingredient or includes only a nominal amount of that particular ingredient.

The polymeric quaternary ammonium compounds useful in the compositions of the present invention are those which have an antimicrobial effect and which are ophthalmically acceptable. Preferred compounds of this type are described in U.S. Pat. Nos. 3,931,319; 4,027,020; 4,407,791; 4,525,346; 4,836,986; 5,037,647 and 5,300,287; and PCT application WO 91/09523 (Dziabo et al.). The most preferred polymeric ammonium compound is polyquaternium 1, otherwise known as POLYQUAD® or ONAMERM® with a number average molecular weight between 2,000 to 30,000. Preferably, the number average molecular weight is between 3,000 to 14,000.

The polymeric quaternary ammonium compounds are generally used in the compositions of the present invention in an amount that is greater than about 0.00001 w/v %, more typically greater than about 0.0003 w/v % and even more typically greater than about 0.0007 w/v % of the ophthalmic composition. Moreover, the polymeric quaternary ammonium compounds are generally used in the compositions of the present invention in an amount that is less than about 3 w/v %, more typically less than about 0.003 w/v % and even more typically less than about 0.0015 w/v % of the ophthalmic composition.

The present invention is directed also to a thermal stable ophthalmic emulsion, the emulsion consisting essentially of or consisting of:
water forming an aqueous phase;
oil forming an oil phase, wherein the oil in amount from 0.8 to 1.2 w/v %;
an ionic charged phospholipid, wherein the ionic charged phospholipid in amount of 0.005 w/v %;
a hydrophilic surfactant, wherein the hydrophilic surfactant is polyoxyethylene-40-stearate and in amount of 0.5 w/v % to 0.8 w/v %.
a hydrophobic surfactant, wherein the hydrophobic surfactant is sorbitan tristearate and in amount of 0.4 w/v % to 0.6 w/v %.
borate, wherein the borate is a boric acid in amount of 1.0 w/v %;
sodium hyaluronate, wherein the sodium hyaluronate is in amount of 0.15 w/v %;
galactomannan polymer, wherein the galactomannan polymer is in amount of 0.15 w/v
wherein the thermally stable ophthalmic emulsion increase of D90 less than 20% after 6 freeze-thaw cycles.

The emulsion of the present invention can include any of a multitude of ophthalmic therapeutic agents. Non-limiting examples of potential ophthalmic therapeutic agents for the present invention include: anti-glaucoma agents, anti-angiogenesis agents; anti-infective agents; anti-inflammatory agents; growth factors; immunosuppressant agents; and anti-allergic agents. Anti-glaucoma agents include beta-blockers, such as betaxolol and levobetaxolol; carbonic anhydrase inhibitors, such as brinzolamide and dorzolamide; prostaglandins, such as travoprost, bimatoprost, and latanoprost; seretonergics; muscarinics; dopaminergic agonists. Anti-angiogenesis agents include anecortave acetate (RETAANE™, Alcon™ Laboratories, Inc. of Fort Worth, Tex.) and receptor tyrosine kinase inhibitors (RTKi). Anti-inflammatory agents include non-steroidal and steroidal anti-inflammatory agents, such as triamcinolone actinide, suprofen, diclofenac, ketorolac, nepafenac, rimexolone, and tetrahydrocortisol. Growth factors include EGF or VEGF. Anti-allergic agents include olopatadine and epinastine. The ophthalmic drug may be present in the form of a pharmaceutically acceptable salt.

The present invention can be particularly useful for delivery therapeutic agents that relieve symptoms of dry eye conditions. Examples include, without limitation, steroidal and/or non-steroidal anti-inflammatory agents; selective PDE IV inhibitors such as cilomilast, cyclosporins, combinations thereof or the like. The emulsion of the invention can also be used in other fields, such as to deliver cooling agents, deliver antioxidants (omega-3 and omega-6 fatty acids) and other bioactivies for ophthalmic uses. For example, nutriceuticals such as vitamin A (retinol), vitamin D (calciferol), vitamin E, tocopherols, vitamin K (quinone), beta-carotene (pro-vitamin-A) and combinations thereof.

Generally, amounts of therapeutic agent, when used, can be quite variable depending upon the agent or agents used. As such, the concentration of therapeutic agent can be at least about 0.005 w/v %, more typically at least about 0.01 w/v % and even more typically at least about 0.1 w/v %, but typically no greater than about 10 w/v %, more typically no greater than about 4.0 w/v %, still more typically no greater than about 2.0 w/v %.

The emulsions of the present invention may optionally comprise one or more additional excipients and/or one or more additional active ingredients. Excipients potentially used in the ophthalmic emulsions include, but are not limited to, demulcents, tonicity agents, preservatives, chelating agents, buffering agents, and surfactants. Other excipients comprise solubilizing agents, stabilizing agents, comfort-enhancing agents, polymers, emollients, pH-adjusting agents and/or lubricants.

The emulsion is typically aqueous and therefore includes a substantial amount of water, which is typically purified. The emulsion typically includes water at a concentration of at least about 50 w/v %, more typically at least about 85 w/v % and even more typically at least about 93 w/v %, but typically no greater than about 99.99 w/v %, more typically no greater than about 99.0 w/v %, still more typically no greater than about 0.3 w/v % and even still more typically no greater than about 98 w/v %.

The emulsion of the present invention may be formed using a variety of combining and mixing protocol and techniques known to those skilled in the art. According to one preferred embodiment, however, the ingredients are mixed and combined according to a specific protocol. In such protocol, multiple admixtures are formed and those admixtures are combined to form the emulsion. The following lab compounding process was used as starting point:

### 1. Emulsion Preparation

Weigh mineral oil and heat to approx. 70° C. Add polyoxyl 40 stearate and sorbitan tri-stearate in mineral oil, mix until completely dissolved.
Weigh pH adjusted WFI water and heat to approx. 70° C. Add DMPG-Na, mix until completely dissolved.
Combine aqueous and oil phases and mix until homogenous.
Homogenize the above homogenous mixture.
Transfer emulsion to Microfluidizer and process.

### 2. HP Guar Solution Preparation and Sterilization

Collect pH adjusted WFI in a depyrogenated glass. While mixing using overhead mixer, add HP guar slowly, until the HP guar is fully hydrated and homogeneous.
Perform polish filtration on the HP Guar solution and transfer an appropriate amount to the final compounding vessel.
Add appropriate amount of emulsion and mix until homogenous.
Autoclave HP guar and emulsion mixture at 121° C. for 20 minutes using pre-set liquid cycle.
Afterwards bring the formulation to room temperature before proceeding to the next step.

### 3. HA Solution Preparation and Sterilization

Collect pH adjusted WFI in a depyrogenated glass. While mixing at approx. 400 rpm using overhead mixer, add HA slowly, mixing for NLT 4 hours or until the HA is fully hydrated and homogeneous.
Filter the solution through a 0.22 μm Sterile filtration unit.
Transfer and cap the bottle in the Horizontal Laminar Flow Hood.

### 4. Salt Solution Preparation and Sterilization

Collect approx. 10% batch wt. WFI in a depyrogenated glass. Heat to 70° C. and add boric acid, sorbitol, and propylene glycol.
Stir at 600 RPM until dissolved.
Allow to cool to RT and adjust pH to 6.1 (6.0-6.2) using 6N Sodium Hydroxide.

Sterile filter through 0.22 μm filter and transfer to the laminar flow hood

### 5. Final Compounding

Addition of HA and salt solution utilizing aseptic techniques and final compounding.
Add the HA solution to the HP Guar/Emulsion solution via aseptic pumping.
Add the salt solution to the formulation via aseptic pumping then mix until homogenous.
Using IN Sodium Hydroxide or IN Hydrochloric acid, adjust the pH of the formulation to 7 (6.5-7.5).
QS the formulation to 100% with WFI and mix until homogenous.

Perform bulk formulation visual inspection, to check for any particulates and phase separation.

Advantageously, the stability of the oil in water emulsion of the present invention can facilitate lubrication and/or the delivery of lipids (e.g., lipid therapeutic agents) to the ocular surface. These lipids can aid in stabilizing the tear film and/or can provide alternative therapeutic advantages to the eye. Moreover, the mucoadhesive polymer can aid residence time of the emulsions upon the eye such that the emulsions can be more efficacious.

### Example 1

A lab-built droplet-based evaporimeter system with addition of environmental control system was used to measure in vitro evaporation rate of two oil in water emulsion formulation samples (FID 122505 and FID 123301).

The customized evaporimeter system based on drop-shape analysis consist of a goniometer with dispensing syringe pump (Rame'-Hart Model 590-U4, Succasunna, NJ, USA) and accompanying controller software DropImage (version 3.23.05.0). The dispensing capillary terminates into a closed chamber that surrounds the pinned sessile droplet. Temperature and humidity are controlled (Rame'Hart Model 100-26-TH, Succasunna, NJ, USA) allowing the environment to be set at desired levels.

This evaporation rate measurement can be used to evaluate the effectiveness of eye-drop formulations in reducing tear evaporation, which is important for managing dry eye.

The results for examples of oil in water emulsion formulation samples with their compositions for Evaporative flux (μm/min), Creaming Rate (mm/day), Particle Diameter (D90), nm (Anton Paar) and D90 increase, %, after freeze-thaw cycling are provided in Table 1.

TABLE 1

| | FID 122505 | FID 123299 | FID 123300 | FID 123301 | FID 12330x |
|---|---|---|---|---|---|
| COMPONENT | w/v %. | w/v %. | w/v %. | w/v %. | w/v %. |
| HP-Guar | 0.15 | 0.15 | 0.15 | 0.15 | 0.15 |
| Sodium | 0 | 0.15 | 0.15 | 0.15 | 0.15 |
| hyaluronate | | | | | |
| Mineral oil | 1.00 | 1.00 | 1.00 | 1.00 | 1.00 |
| Boric Acid | 1.00 | 1.00 | 1.00 | 1.00 | 1.00 |
| Anionic | 0.005 | 0.005 | 0.005 | 0.005 | 0.005 |
| Phospholipid | | | | | |
| Polyoxyl 40 | 0.38 | 0.38 | 0.76 | 0.57 | 1.14 |
| Stearate (P40) | | | | | |
| Sorbitan | 0.29 | 0.29 | 0.58 | 0.435 | 0.87 |
| Tristearate | | | | | |
| (Span 65) | | | | | |
| Propylene Glycol | 0.6 | 0.6 | 0.6 | 0.6 | 0.6 |
| Sorbitol | 0.7 | 0.7 | 0.7 | 0.7 | 0.7 |
| Sodium Hydroxide | Adjust pH | Adjust pH | Adjust pH | Adjust pH | Adjust pH |
| | to 7.0 | to 7.0 | to 7.0 | to 7.0 | to 7.0 |
| Hydrochloric Acid | Adjust pH | Adjust pH | Adjust pH | Adjust pH | Adjust pH |
| | to 7.0 | to 7.0 | to 7.0 | to 7.0 | to 7.0 |
| Purified Water | QS 100 | QS 100 | QS 100 | QS 100 | QS 100 |
| (HP-Guar + HA)/ | 0.22 | 0.45 | 0.22 | 0.30 | 0.15 |
| (P40 + Span65 | | | | | |
| Oil/(P40 + Span 65) | 1.49 | 1.49 | 0.75 | 1.0 | 0.5 |
| Evaporative flux | 7.6 | | | 6.7 | |
| (μm/min) | | | | | |
| Creaming Rate | 0.114 | 0.639 | 0.073 | 0.232 | 0.044 |
| (mm/day) | | | | | |
| Particle | 112 | 127 | 89 | 103 | 88 |
| Diameter(D90), nm | | | | | |
| (Anton Paar) | | | | | |
| D90 increase, %, | 19 | 79 | 11 | 7 | 43 |
| after freeze-thaw | | | | | |
| cycling | | | | | |

We claim:

1. A thermal stable ophthalmic emulsion, the emulsion comprising:
water forming an aqueous phase;
oil forming an oil phase, wherein oil in amount from 0.8 to 1.2 w/v %;
a charged phospholipid;
two surfactants, a first surfactant is a hydrophilic surfactant having an HLB value of from 10 to 18, a second surfactant is a hydrophobic surfactant having an HLB value of from 1 to 6, and a combination thereof, wherein the first surfactant is not the charged phospholipid;
borate;
two mucoadhesive polymers, the first mucoadhesive polymer is sodium hyaluronate, the second mucoadhesive polymer is galactomannan polymer;
wherein a ratio of the total concentration of the two mucoadhesive polymers to the total concentration of two surfactants is between 0.20 and 0.40;
Wherein a ratio oil to the total concentration of two surfactants is between 0.6 and 1.1;
wherein the thermally stable ophthalmic emulsion increase of D90 less than 40% after 6 freeze-thaw cycles
The borate and galactomannan polymer cooperatively act to form a gel upon instillation of the emulsion in an eye of an individual.

2. The emulsion according to claim 1 wherein the oil is a hydrocarbon selected from mineral oil, paraffin oil and petrolatum.

3. The emulsion according to claim 1 wherein the oil is at least 0.9 w/v % and no greater than 1.1 w/v % of the emulsion.

4. The emulsion according to claim 1 wherein the hydrophilic surfactant is present in the emulsion in an amount between 0.15 w/v % to 1.0 w/v %.

5. The emulsion according to claim 1 wherein the hydrophilic surfactant is polyoxyethylene-40-stearate.

6. The emulsion according to claim 1 wherein the hydrophobic surfactant is present in the emulsion in an amount between 0.15 w/v % to 1.0 w/v %.

7. The emulsion according to claim 1 wherein the hydrophobic surfactant is a sorbitan tristearate.

8. The emulsion according to claim 1 wherein the mucoadhesive galactomannan polymer is selected from the group consisting of guar and hydroxypropyl guar.

9. The emulsion according to claim 1 wherein the mucoadhesive galactomannan polymer is present in the emulsion at a concentration of at least 0.01 w/v %, but no greater than 1.0 w/v.

10. The emulsion according to claim 1 wherein the mucoadhesive sodium hyaluronate is present in the emulsion at a concentration of at least 0.01 w/v %, but no greater than 1.0 w/v.

11. The emulsion according to claim 1 wherein the charged phospholipid is an anionic phospholipid named dimyristoyl phosphatidylglycerol.

12. The emulsion according to claim 1 wherein the phospholipid is present in the emulsion in a concentration from 0.05 to 7.0 percent by weight.

13. The emulsion according to claim 1 further comprising a borate/polyol buffer system.

14. Then emulsion according to claim 1, further comprising a preservative, wherein the preservative is a polymeric quaternary ammonium compound.

15. A thermal stable ophthalmic emulsion, the emulsion consisting essentially of or consisting of:
water forming an aqueous phase;
oil forming an oil phase, wherein the oil in amount from 0.8 to 1.2 w/v %;
an ionic charged phospholipid, wherein the ionic charged phospholipid in amount of 0.005 w/v %;
a hydrophilic surfactant, wherein the hydrophilic surfactant is polyoxyethylene-40-stearate and in amount of 0.5 w/v % to 0.8 w/v %.
a hydrophobic surfactant, wherein the hydrophobic surfactant is sorbitan tristearate and in amount of 0.4 w/v % to 0.6 w/v %.
borate, wherein the borate is a boric acid in amount of 1.0 w/v %;
sodium hyaluronate, wherein the sodium hyaluronate is in amount of 0.15 w/v %;
galactomannan polymer, wherein the galactomannan polymer is in amount of 0.15 w/v
wherein the thermally stable ophthalmic emulsion increase of D90 less than 20% after 6 freeze-thaw cycles.

16. The emulsion according to claim 15, wherein the oil in amount of 1.0 w/v %;
wherein the polyoxyethylene-40-stearate is in amount of 0.76;
wherein the sorbitan tristearate is in amount of 0.58.

17. The emulsion according to claim 15, wherein the oil in amount of 1.0 w/v %;
wherein the polyoxyethylene-40-stearate is in amount of 0.57;
wherein the sorbitan tristearate is in amount of 0.435.
